(19) United States
(12) Patent Application Publication
(10) Pub. No.: US 2007/0196308 A1
(43) Pub. Date: Aug. 23, 2007

(54) Antiperspirant clear gel with low glycol content

(76) Inventors: Christine Popoff, Morganville, NJ (US); Marian Holerca, Somerset, NJ (US); Diane Henao, Dover, NJ (US); John Brahms, Morris Plains, NJ (US)

Correspondence Address: COLGATE-PALMOLIVE COMPANY, 909 RIVER ROAD, PISCATAWAY, NJ, 08855, US

(21) Appl. No.: 10/571,488

(22) PCT Filed: Sep. 8, 2004
(86) PCT No.: PCT/US04/29116

§ 371 Date: Apr. 2, 2007

Related U.S. Application Data
(60) Provisional application No. 60/501,128, filed on Sep. 8, 2003.

(51) Int. Cl. A61K 8/89

(52) U.S. Cl. 424/70.12

(57) ABSTRACT

An elastomer-free, suspension-free, water-in-oil emulsion as a clear gel with a viscosity >150,000 centipoise; and an overall level of silicone emollients ≦3 weight % as: (a) an internal phase comprising a glycine-free antiperspirant active stabilized by Betaine; and a glycol system having ≦7.5 weight % propylene glycol; and (b) an external phase comprising cyclomethicones; a silicone copolyol; and a fragrance solubilizer; wherein: the maximum level of volatile linear silicones is ≦1 weight %, the water content is >30 weight %, and the external phase is free of silicone emollients with a refractive index >1.4200.

### FIELD OF THE INVENTION

The present invention is directed to a clear, high efficacy, high viscosity, elastomer-free, suspension-free gel composition which comprises a glycine free antiperspirant active (preferably with a lower metal to chloride ratio) stabilized by trimethylglycine (“Betaine”) and containing low amounts of glycols and low levels of nonvolatile ingredients. The active phase may additionally contain a monovalent or divalent ionizable water soluble salt. The gel is formulated as a clear product having reduced whitening and tack as well as reduced skin irritation. An odor neutralizing agent which provides improved odor protection is an additional optional ingredient which can easily be incorporated within the gel composition.

### BACKGROUND OF THE INVENTION

U.S. patent application Ser. No. 10/406,856 filed Apr. 4, 2003, comprises aluminum and/or zirconium salts with Betaine as a complexing agent and buffering agent and which do not contain glycine. Betaine can be used in its normal form or as Betaine hydrochloride. The enhanced salts of this invention may be used to formulate antiperspirants having improved efficacy. Such antiperspirants include solids such as sticks and creams (creams sometimes being included in the term “soft solid”), gels, liquids (such as are suitable for roll-on products), and aerosols. The forms of these products may be suspensions or emulsions.

U.S. patent application Ser. No. 10/607,099 filed Jun. 26, 2003, comprises aluminum/zirconium salts containing a complexation agent such as glycine and further stabilized with Betaine as complexation agent and buffer agent. The amount of Betaine that is used should be sufficient to have an overall Betaine+glycine to Zr ratio in the range of 0.1-3.0:1, and preferably in the range of 0.7-1.5:1, with a ratio of Betaine to glycine of at least 0.001:1. The enhanced salts of this invention may be used to formulate antiperspirants having improved efficacy.

Antiperspirant products are well known in the art. Antiperspirants have appeared in the marketplace in varied dosage forms, such as sticks, soft solids, soft gels, roll-on, aerosols and creams. Generally, these dosage forms include a solution of the active ingredient in a suitable solvent, a suspension of the active ingredient in a non-solvent, or a multiphasic dispersion or emulsion in which a solution of the active ingredient is dispersed in some continuous phase or in which the solubilized active ingredient constitutes a continuous phase.

Of the above-referred-to dosage forms, the stick form is an example of a solid form, and the soft solid and soft gel are thickened forms which may or may not be solid (for example, under some circumstances, gels can flow). The stick form can be distinguished from a soft solid or soft gel in that, in a stick, the formulated product can retain its shape for extended time periods outside the package, the product not losing its shape significantly (allowing for some shrinkage due to solvent evaporation). Adjustment of amounts of gelling or thickening agents can be used in order to form a soft gel or stick.

Soft gels or soft solids can be suitably packaged in containers which have the appearance of a stick, but which dispense through apertures (for example, slots or pores) on the top surface of the package. The soft solid products have also been called soft sticks or “smooth-ons”, and hereinafter are generically called “soft solids”. Reference is made to U.S. Pat. No. 5,102,656 to Kasat, U.S. Pat. No. 5,069,897 to Orr, and U.S. Pat. No. 4,937,069 to Shin, each of which discloses such soft solids, including physical characteristics thereof such as viscosity and hardness. The contents of each of these three U.S. patents are incorporated herein by reference in their entirety for description of characteristics of soft solids and suitable packaging for such products.

Recently, there has been significant activity in developing clear antiperspirant sticks and soft gels, particularly to provide sticks and soft gels having increased efficacy (for example, by providing increased amounts of the antiperspirant active in the sticks and soft gels), improved cosmetic characteristics (including reduced whitening, reduced residue and reduced tack), and reduced skin irritation potential (e.g., providing a product that is “mild”).

U.S. Pat. No. 4,944,938 to Potini discloses clear, non-alcoholic, quick drying, antiperspirant and deodorant gels, which are stable both at room temperatures and at higher temperatures, are non-stinging and leave no white residue on the skin. The gel is free of gelling agents, waxes, clays, or monohydric alcohols having 2-8 carbon atoms. The gels use 3-5 carbon atom trihydric alcohols as coupling agents which act as solubilizers in the system and keep the system stable and clear. The gels can include an aluminum active salt; a volatile water-insoluble emollient, such as isostearyl benzoate: a soluble emollient such as cetyl ether; solubilizers such as propylene glycol and glycerin; volatile siloxanes; and water.

Some cellulosic materials, such as hydroxypropylcellulose, among others, are compatible with polyvalent metal salts and have been used in the manufacture of clear lotions. These cellulosic materials, however, must be prepared with a high percentage of water or alcohol in order to insure solubilization of the active ingredient. The resulting formulations, in addition to a high irritation potential, are tacky and low in efficacy, when alcohol-based; and exhibit tackiness and along drying time when water-based.

Clear antiperspirant soft gels (which have been dispensed from containers having the appearance of stick) have recently been marketed, consisting of viscous, high-internal-phase emulsions. These soft gels exhibit some advantages over the aforementioned sticks, particularly acetal-based clear sticks, in that the selection of formulation ingredients is less restricted (for example, water can be used), and often tack can be reduced significantly. Concerning these emulsions, note U.S. Pat. No. 4,673,570 to Soldati and U.S. Pat. No. 4,900,542 to Parrotta, et al. These two U.S. patents disclose clear gelled antiperspirant compositions free of waxes and conventional gelling agents, containing a volatile silicone fluid, a silicone emulsifier, a destabilizing auxiliary emulsifier, water, non-volatile emollient, a coupling agent, an active antiperspirant component and ancillary agents such as perfume, coloring agents, etc. The silicone emulsifiers a cyclomethicone-dimethicone copolyol silicone fluid marketed by Dow Corning Corporation under the trademark DOW CORNING 3225C formulation. In particular, U.S. Pat. No. 4,673,570 claim 1 is “from about 10-25% by weight of a cyclomethicone-dimethicone copolysilicone fluid (same as 1.0-2.5% on an actives basis). The contents of these two U.S. patents are incorporated herein by reference in their entirety with respect to the features of the inventions described therein.

Also to be noted is PCT (International application) Publication No. WO 92/05767. This patent document discloses a clear gel-type cosmetic product having a viscosity of at least about 50,000 cps at 21 degrees C. and a refractive index of 1.3975-1.4025 at 21 degrees C., and having an optical clarity better than 50 NTU (Nephelometric Turbidity Units) at 21 degrees C., the product being an emulsion with a water phase having an active ingredient incorporated therein and with an oil phase. The refractive indices (measured at 5893 Angstroms) of the water and oil phases match to within 0.0004. The oil phase includes an emulsifier which when properly mixed with the water phase component yields a water-in-oil emulsion, and the water phase includes one or a combination of various polar species such as water, propylene glycol, sorbitol and ethanol. The water phase includes the deodorant and/or antiperspirant active ingredient. The contents of this PCT (International application) Publication No. 92/05767 are incorporated herein by reference in their entirety.

U.S. Pat. No. 6,007,799, assigned to the same owner as this case, describes clear cosmetic gels that are water-in-oil emulsions and which comprise at least one coupling agent, silicone fluids and an alkoxylated, alkyl substituted silicone surface active agent.

U.S. Pat. Nos. 5,587,153 and 5,863,525 issued to Gillette also describe gel products that (1) contain silicone in the oil phase and (2) does not control the propylene glycol content.

U.S. Pat. No. 5,925,338 issued to Gillette describes a clear gel comprising selected amounts of various types of silicones.

U.S. Pat. No. 6,419,910 assigned to Unilever describes a clear emulsion and gel-type antiperspirant and deodorant composition which comprises a water in oil emulsion which is essentially free of glycols and low and middle chain alcohols. The composition comprises 25-35% of an oil phase comprising at least one non-volatile ester or at least one nonvolatile silicone wherein at least one oil phase soluble ingredients has a refractive index of about 1.40 to about 1.45. The active phase contains a water soluble, non-simple glycol component which raises the refractive index of the aqueous solution.

U.S. Pat. No. 6,410,002 and U.S. Patent Application 2002/10051138 A1 also assigned to Unilever, describes an essentially glycol free clear emulsion and gel-type antiperspirant and deodorant composition in which the water phase further differentiated by containing at least one polymeric ethylene oxide glycol and is essentially free of glycols and low and middle chain alcohols.

U.S. Patent Application Publication No. 2004/0001795 A1 (Publication Date: Jan. 1, 2004) filed by Reheis describes a glycine free high efficacy antiperspirant salt comprised of aluminum zirconium tetrasalt with a low M:Cl ratio.

U.S. patent application Ser. No. 10/625,038 filed by Reheis describes a glycine free high efficacy antiperspirant salt comprised of aluminum zirconium octasalt with a low M:Cl ratio.

Further examples of making these types of salt complexes are described in U.S. patent application Ser. No. 10/406,856, filed Apr. 4, 2003 and Ser. No. 10/607,099, filed Jun. 26, 2003.

U.S. Pat. No. 6,042,816 describes enhanced efficacy antiperspirant salt compositions containing calcium and an amino acid or a hydroxy acid, methods of making such enhanced efficacy antiperspirant salt compositions, stabilized aqueous solutions of such enhanced efficacy antiperspirant salt compositions, and topical compositions containing such enhanced efficacy antiperspirant salt compositions.

U.S. Pat. No. 6,468,512 assigned to Avon describes a clear antiperspirant/deodorant gel composition. The composition is a water-in-oil emulsion having a viscosity about 7,000 cps to about 25,000 cps and a clarity from about 30 NTU or less. The composition further has an antiperspirant active, water, silicone gelling agent, and one or more silicone oils.

U.S. Pat. No. 6,485,716 assigned to the same owner as the present case describes a clear, elastomer-free, gel composition comprising: (a) 0.1-25 weight % of an antiperspirant active having a low metal to chloride ratio in the range of 0.9-1.3:1; (b) 9-23.95 weight % of one or more volatile silicones having a flash point of 100 degrees C. or less; (c) 0.05-0.5 weight % of a silicone surfactant having an HLB value less than or equal to 8; (d) 30-70 weight % water; (e) 0-50 weight % selected water soluble organic solvents; and (f) 0-10 weight % of an emollient; wherein the composition is a liquid gel having a viscosity in the range of 5-50,000 centipoise and a ratio of oil phase to water phase in the range of 10:90 to 24:76.

U.S. Pat. No. 6,500,412 assigned to the same owner as this case describes a non-sticky, clear water-in-oil emulsion comprising: (a) 65-90 weight % of an internal phase comprising 5-35 weight % of an antiperspirant salt (anhydrous basis) having a metal:chloride ratio in the range of 0.9-1.4:1; 5-15 weight % of tripropylene glycol; and 35-70 weight % water; and (b) 10-35 weight % of an external phase comprising 1-40 weight % of a volatile silicone which is not an elastomer; 0.1-5 weight % of a silicone copolyol surfactant; and 0-20 weight % of a nonvolatile silicone which is not an elastomer; wherein the composition is free of (1) C1-5 saturated alcohols, (2) added propylene glycol, (3) elastomer gelling agents, (4) soap gelling agents (5) borate gelling agents, and (6) coupling agents, and wherein all amounts are in % by weight based on the total weight of the composition.

The Betaine (defined below) of this invention, is not a surfactant and has been found to have properties important to the field of antiperspirant salts that contain zirconium. The Betaine used in this invention is a natural product found in a number of plants in the Chenopodiaceae family, and also in fish and selected legumes. Extracted most often from sugar beets (Beta Vulgaris), it is reported as an extremely versatile molecule with a wide range of applications: food supplement, anti-irritant, skin moisturizer, skin-softening agent, skin-conditioning agent, promoter of wound healing and component in cosmetic compositions for skin aging and stressed skin.

Betaine in IUPAC nomenclature is 1-carboxy-N,N,N-trimethylmethanaminium hydroxide-inner salt, with alternative names including carboxymethyl-trimethyl-ammonium betaine or (carboxymethyl)trimethylammonium hydroxide-inner salt or glycine betaine or glycol betaine or glycyl betaine or trimethylglycine or trimethylglycol. For convenience here the material of Formula I (C₅H₁₁NO₂; Mass=117.08 amu; molecular weight=117.15; analysis as C, 51.26; H, 9.46; N, 11.96; O: 27.32) will be referred to as Betaine.

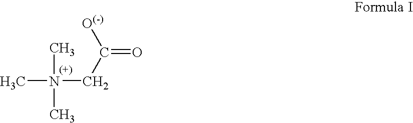

Betaine appears in numerous patents, with a wide range of applications.

German Patent DE 19725087 is related to cosmetic and dermatologic oil-in-water emulsion formulations for light protection containing hydrophobic inorganic micropigments and hydrophilic surfactants.

PCT Publication WO 97/23594 describes skin cleansing compositions with enhanced antimicrobial activity comprising 0.1-30% of an amphoteric, zwitterionic, nonionic, anionic and/or cationic emulsifier, 0.00001-5% of an Ag compound (AgCl, Ag₂CO₃, etc.), deposited on a particulate inert support material (metal oxides, especially TiO₂) as antimicrobial agent, and H₂O. A typical composition contains cetyl betaine.

Japanese Patent JP 2001163752 describes long-lasting cosmetic makeup compositions comprising plate-type glossy polymer powders and antiperspirants.

European Patent EP 1005853 describes the use of betaines as antiperspirants. Mono-, di-, and trimethylammonio-substituted carboxylic acids R¹R²R³N+(CH₂)ₙC(O)O— (R¹-R³=H, Me; n=1-10) are active as antiperspirants and are compatible with the skin and with other conventional constituents of antiperspirant and deodorant compositions.

European Patent EP 1005852 describes the use of functionally substituted betaines as antiperspirants. Mono-, di-, and trimethylammonio-substituted carboxylic acids R¹R²R³N+(CH₂)ₙCHX(CH₂)ₘC(O)O— and/or X(CH₂)ₙCH(N+R¹R²R³)(CH₂)ₘC(O)O— (R¹-R³=H, Me; m, n=1-8) are active as antiperspirants and are compatible with the skin and with other conventional constituents of antiperspirant and deodorant compositions.

Japanese Patent JP 11130652 discloses skin-conditioning and moisturizing cosmetics containing clay minerals and low-molecular-weight betaines to inhibit the release of pyrrolidinecarboxylic acid (a natural moisturizing factor) from human skin.

German Patent DE 2610225 describes aluminum salts of Betaine chloride being useful as ulcer inhibitors, for treatment of gastritis, to promote wound healing, and as antiperspirants and deodorants.

PCT Publication WO 01/62222 describes cosmetic compositions containing phospholipids and quaternary amines. The invention relates to a cosmetic composition, especially for use on aging and/or stressed skin, the composition comprising, in addition to water, at least one substance that forms lamellar structures with water. Compositions including Betaine are described.

PCT Publication WO 01/47479 assigned to the same owner as this case describes cosmetic moisturizing compositions containing quaternary ammonium compounds. Compositions with cocamidopropyl betaine are described.

PCT Publication WO 01/39730 describes a cosmetic composition containing peat and Betaine.

PCT Publication WO 97/46246 is related to complex preparations for topical use containing Betaine to stimulate cellular and physiological processes.

PCT Publication WO 91/18588 presents a method of reducing the irritating properties of a cosmetic composition by addition of Betaine derivatives.

Japanese Patent JP 03033266 describes modified fabrics coated with a mixture of dodecyl betaine and other ingredients for controlling pH change in skin during sweating.

While various cosmetic gel compositions, including antiperspirant and deodorant compositions that are clear are known, it is still desired to provide a cosmetic gel composition (e.g., clear antiperspirant and/or deodorant gel composition) which has improved efficacy in comparison to other products, especially other commercially available gel products. Betaine, is able to further stabilize the antiperspirant salts and thus maintain or boost their efficacy in time.

With regard to odor masking and neutralizing technologies a number of materials have been reported to have malodor counteracting properties alone or in combination with fragrances. The modes of action of these materials and compositions fall into several general classes which include (a) fragrant materials or precursors thereof which mask the perception of malodorous materials, (b) materials and compositions that kill bacteria or disrupt processes by which bacteria on the skin or in the environment convert non-odorous materials to malodorous volatile compounds, (c) materials and compositions that suppress the volatility of offending materials through physical adsorption or absorptions or by chemically reacting with malodor volatiles rendering them non-volatile, (d) materials and compositions that, while not highly odorous in their own right, mask the chemoreception of malodorous materials by the human nose. Much of the prior art is related to reducing malodors by utilizing a combination of one or more of the above mentioned mechanisms.

In U.S. Pat. No. 6,495,097, Streit et. al disclose a composition and a method of use for reducing malsensory agents comprising a component selected from the group consisting of fragrances, flavors, unfragranced carriers and mixtures thereof, with undecylenic acid and/or a derivative thereof, in an amount effective to reduce the malsensory agents and allow release of the component from the composition.

It is known that fragrances may be designed to counteract malodors. The fragrance materials, which are most common to mask a malodor are those that contain a carbon-carbon double bond conjugated with one or more carbonyl groups. Aldehydes are the most commonly used materials of this class for counteracting malodors, with those most commonly used for deodorant properties being trimethyl hexanal, other alkyl aldehydes, benzaldehyde, and vanillin. For example, European Patent Application 0404470 discloses the use of fragrance materials with good malodor reduction efficacy, and European Patent Application 0545556 discloses mixtures of fragrance materials that mask malodors. The use of fragrance materials alone, however, may limit the types of fragrances a perfumer can create.

Other materials have also been shown to have malodor counteractant (“MOC”) properties. Schleppnik, U.S. Pat. No. 4,622,221 (“Schleppnik '221”) discloses the use of cyclohexyl alcohols and ester derivatives in room fresheners. Kulka, U.S. Pat. No. 3,074,891 discloses esters of alpha-, beta-unsaturated monocarboxylic acids as malodor counteractants. Kulka, U.S. Pat. No. 3,077,457 discloses fumaric acid esters as malodor counteractants. Schleppnik, U.S. Pat. No. 4,187,251 discloses alkyl cyclohexyl alkyl ketones as malodor counteractants. Schleppnik, U.S. Pat. No. 4,310,512 discloses the use of derivatives of acetic and propionic acids, and Schleppnik et al., U.S. Pat. No. 4,009,253 discloses the use of 4-cyclohexyl-4-methyl-2-pentanone as a malodor counteractant. These materials, however, are not capable of neutralizing all types of functional groups contained in malodor molecules. All of the U.S. patents discussed above are hereby incorporated by reference as if recited in full herein with respect to the teaching of substances that can counteract malodors.

In U.S. Pat. No. 6,610,648 McGee et. al. disclose malodor counteracting compositions with low odor intensity that contain alpha, beta-unsaturated carbonyl compounds. Although these malodor counteracting compositions reduce the perception of a variety of malodors it is not readily apparent that such compositions may also mask perceived fragrance intensity. The aforementioned patent application envisions the use of malodor counteracting accords in deodorant sprays, soft solids and solids, and antiperspirant sprays, soft solids, and solids and demonstrate inhibition of a synthetic sweat odor by applying an alcoholic solution of said malodor counteracting composition to a cotton pad impregnated with malodor. However no examples are presented or envisioned whereby an in-vivo malodor counteracting effect is demonstrated by delivery on said malodor counteracting composition to the human axilla from a water-in-oil antiperspirant emulsion composition.

While various cosmetic gel compositions, including antiperspirant and deodorant compositions that are clear are known, it is still desired to provide a cosmetic gel composition (e.g., clear antiperspirant and/or deodorant gel composition) which has improved efficacy in comparison to other products, especially other commercially available gel products.

According to a first aspect of the present invention, various of the foregoing objects are achieved through a high viscosity emulsion having (1) an aqueous phase containing water (or water and a water soluble organic solvent as defined above); the antiperspirant active containing aluminum and zirconium metals having a low M:Cl ratio (0.9 to 1.3:1) and stabilized by Betaine; and (2) an oil phase containing a volatile organic or silicone material, and the composition further including (3) a suitable silicone based surfactant such as an alkoxylated, alkyl substituted siloxane surface active agent in an amount of 0.6-0.9 weight % (on an actives basis) which suitable to form a high viscosity (>150,000 cps) gel as described above and (4) a suitable fragrance solublizer such as myristyl ether which helps to solubilize the fragrance oils in the predominantly silicone based oil phase.

It is a further object of the invention to provide products which have (a) reduced whitening, (b) low tack, (c) a quick dry down profile and (d) reduced skin irritation potential relative to commercially available products. It is yet another object of the invention to provide gel antiperspirant/deodorant products which are free of oil soluble high refractive index (>1.420) emollients (which tend to slow down drying time of the gel composition due to their low volatility). This invention has an oil phase which has a relatively low refractive index when compared to other clear gels, thereby reducing the level of water soluble organic or silicone based refractive index matching agents (such as glycols and other monohydric or polyhydric alcohols, ionizable monovalent or divalent inorganic salts, sugars, esters and amino acids) used to match the refractive index of the water (internal) phase to the oil (external) phase to obtain a clear gel.

It is a further object of this invention to provide antiperspirant and/or deodorant compositions which are not as sensitive to fragrance degradation as other products with low metal:Cl ratios which might be expected to degrade fragrances. Formulas of this invention being free of buffering agents that contain nucleophilic amino groups such as glycine exhibit improved stability of (1) fragrances and (2) odor neutralizing alpha, beta unsaturated carbonyl ester compounds.

### SUMMARY OF TH INVENTION

The compositions of this invention may be used as antiperspirants and/or deodorants. The invention is a clear gel (50-250 NTU at 21.0 degrees C.); high viscosity (>150,000 centipoise); elastomer-free; suspension-free; water-in-oil emulsion having an overall level of silicone emollients ≦3 weight % wherein the clear gel comprises:

(a) an internal (water) phase comprising:

(i) 14-30 weight % of a glycine-free antiperspirant active with a low metal to chloride ratio in the range of 0.9-1.3:1 and stabilized by Betaine;

(ii) a high water content such that the water level of the entire composition is >30 weight % water based on the total gel composition; and

(iii) 3.5-10 weight % of a glycol system comprising propylene glycol and a second glycol selected from the group consisting of glycols and polyglycols in which the total amount of propylene glycol in the total gel composition is ≦7.5 weight %; and

(b) an external (oil) phase comprising:

(i) one or more cyclomethicones having a flash point of 100 degrees C. or less (for example, one or more of D4, D5 and D6 cyclomethicones);

(ii) a silicone copolyol; and

(iii) a fragrance solubilizer; and

(iv) 0-0.45 weight % (particularly 0.05 to about 0.45 weight %) of an alpha, beta-unsaturated ester (or mixtures thereof) malodor counteracting material;

(v) 0-3 weight % of a silicone emollient;

wherein: (1) all the amounts are based on the total formulation, (2) the maximum level of volatile linear silicones (if used) is ≦1 weight %, and (3) the external phase is free of silicone emollients that have a refractive index >1.4200.

One particular embodiment is free of amino acids. Another embodiment contains amino acids provided the gel composition is still glycine-free.

These cosmetic gels are quick drying due to the combination of low levels of nonvolatile materials in the oil phase.

Monovalent or divalent salt can be optionally used as refractive index modifying agents in the aqueous phase so as to match the refractive index of the oil phase, thus rendering a clear gel.

The present invention may also contain as an optional ingredient which is at least one malodor counteracting alpha, beta-unsaturated ester or mixtures of such materials. If incorporated within the antiperspirant and/or deodorant composition defined in this invention, the level of malodor counteracting components in the finished formula composition falls within a narrowly defined effective window, the definition of which is not obvious without in-vivo deodorancy assessment. This invention identifies the optimal level of malodor counteracting composition to deliver a perceivable odor control benefit when delivered from an antiperspirant and/or deodorant gel as being in the range of about 0.05 to about 0.45 weight % based on the entire gel composition. The alpha,beta-unsaturated ester malodor counteracting materials are incorporated within the oil phase of the antiperspirant composition. Based on the chemical composition of these malodor counteracting components which include, but are not limited to, those discussed in U.S. Pat. No. 6,610,648 (Givaudan) and U.S. Pat. No. 6,495,097 (Shaw et al), it is not obvious that they should be stable in an antiperspirant and/or deodorant gel containing a higher acidity (low M:Cl) antiperspirant active salt. For example, in this invention the odor neutralizing alpha, beta unsaturated ester mixture demonstrates unexpected stability in the aforementioned antiperspirant compositions containing low metal:chloride (M:Cl) ratio salts free of glycine. In contrast, these malodor counteracting compositions are unstable when formulated within gel, cream and liquid gel compositions comprised of glycine containing antiperspirant active salts wherein the metal/Cl ratio of the salt is low (low being defined for aluminum zirconium tetra or octasalts the wherein the metal/Cl ratio is in the range of 0.9-1.3:1; glycine/Zr ratio is >1.2; for aluminum salts, the aluminum to chloride ratio being in the range of 0.5-2.5:1; the glycine/Al ratio being in the range of 0.05-0.26:1).

### DETAILED DESCRIPTION OF THE INVENTION

The invention is a clear gel (50-250 NTU at 21.0 degrees C.); high viscosity (>150,000 centipoise); elastomer free; suspension-free; water-in-oil emulsion having an overall level of silicone emollients ≦3 weight % wherein the clear gel comprises, for example, an external phase comprising (in percentages are based on overall formula composition):

(a) 6.5-23.4 weight % (more particularly 9-20 weight %) of one or more cyclomethicones having a flash point of 100 degrees C. or less;

(b) 0.6-0.9 weight % on an active basis (particularly 0.6-0.8%) of a silicone surfactant having an HLB value (hydrophilic lipophilic balance)≦8) such as a silicone copolyol (actives basis being the actual amount of the copolyol since the copolyol is frequently found with a carrier such as cyclomethicone is used, for example, as a 10% actives material);

(c) 0.1-3.0 weight % of an non-siliconized organic fragrance solubilizer (particularly 0.5-2%) consisting of silicone compatible straight or branched hydrocarbons with a molecular weight less than 1000, alkyl substituted phenyl esters with an alkyl carbon chain length between C-1 to C-20, and ethoxylated and or propoxylated ethers with a carbon chain length from C-1 to C-25 and a degree of ethoxylation and/or propoxylation from 1-10 (for example, a member of the group consisting of hydrogenated polyisobutene (Polyiso 250), C12-15 alkyl benzoate (FINSOLV TN), and PPG-3 myristyl ether (particularly PPG-3 myristyl ether) which can help to solubilize the fragrance oils in the otherwise all silicone based oil phase and which does not negatively affect the skin feel and dry down characteristics of the composition; (Aesthetic skin feel attributes such as wetness, stickiness, and residue have been evaluated by a 10 member trained panel both on the forearm and underarm by rating some of the antiperspirant gels described in this invention along with a commercial gel. Significant differences in performance between some of the formulas described herein and a commercial gel were noted at the 95% confidence level.)

(d) 0-3 weight % (particularly 0-2.5 weight % or 0.1-1.0 weight %) of a low refractive index (R.I.<1.4200 at 21 degrees C.) silicone emollient (by definition excluding (a) and (b) of this section) selected from the group consisting of (i) volatile linear polydialkylsiloxanes with a flash point is ≦100 degrees C. (particularly low viscosity dimethicones); (ii) nonvolatile linear polydialkylsiloxanes with a flash point >100 degrees C.; (iii) silanols (for example dimethiconols such as DC 9023 and DC 1501 from Dow Corning) in which one or two of the alkyl groups of the foregoing materials (i) or (ii) are replaced with a hydroxyl group; and (iv) mixtures of any of the foregoing (note that combinations of these low refractive index silicone emollients may also be used, however, the maximum level of volatile linear silicone emollients must still be ≦1 weight % (note that one particular embodiment of the invention is free of volatile linear silicones (e.g. volatile dimethicones) and/or free of non-volatile silicones).

(e) 0-5 weight % fragrance or odor masking component; and

(f) 0-0.45 weight % of an alpha, beta-unsaturated ester as a malodor counteracting material.

This external phase may conveniently be combined with an internal phase, for example, an internal phase comprising:

(a) 14-30 weight % on an anhydrous basis (particularly 17-30 weight % and, more particularly, 17-25 weight %) of a glycine-free antiperspirant active salt further stabilized by Betaine comprising either aluminum or aluminum and zirconium metals wherein the choice of components is such that (i) if aluminum and zirconium salt is used then the metal/Cl ratio of the salt should be low, such as 0.9-1.3:1 (more particularly in the range of 0.9-1.05:1); the Betaine/Zr ratio should be within the range of 0.2-3.0:1 (particularly 0.4-1.5:1) and the Betaine:aluminum molar ratio in the range of 0.05-1.0:1 (particularly 0.05-0.26:1 and, more particularly, 0.05-0.16:1); (ii) if only aluminum salt is used the aluminum/Cl ratio of the salt should be within the range of 0.5-2.5:1; the Betaine/Al ratio should be within the range of 0.05-1.0:1; wherein the Betaine stabilized and/or buffered antiperspirant active salt has a pH in the range of 2-4 when measured in water at a concentration of 15% (note that the Betaine serves the dual purpose of both a buffering agent as well as RI index matching agent for the active phase;

(b) 30-70 weight % water (particularly 45-65% and, more particularly, 50-60%);

(c) 3.5-10 weight % particularly 4-8 weight %) of a water soluble glycol system which comprises at least 0.2% and at most 7.5% propylene glycol and an additional glycol component selected from the group consisting of ethylene glycol; diethylene glycol; triethylene glycol; tetraethylene glycol; propylene glycol; dipropylene glycol; tripropylene glycol; 1,3 propanediol; 2-methyl propanediol; methyl propanediol; 1,6-hexanediol; 1,3 butanediol; 1,4 butanediol; PEG-4 through PEG-600; PPG-9 through PPG-34; neopentyl glycol; trimethylpropanediol; 2,2 dimethyl-1,3propandiol; 2,2,4,4-tetramethyl-1,3-cyclobutane-diol; and mixtures thereof in which the amount of propylene glycol does not exceed 7.5% (particularly not exceeding 5%). (More particular examples of the glycol component include one or more members of the group consisting of propylene glycol, dipropylene glycol, 2-methyl-1,3 propanediol, methyl propylene glycol, low molecular weight (less than 600) polyethylene glycol and mixtures of any of the foregoing.)

(d) 0-4 weight % (particularly 0.2-4 weight %) of a monovalent or divalent ionizable, water-soluble inorganic or organic salts to help increase the refractive index of the active phase and optimize the glycol level. These salts are of the form MₐX_{b} where a=1, or 2 and b=1 or 2; M is a member selected from the group consisting of Na⁺¹, Li⁺¹, K⁺¹, Mg⁺², Ca⁺², Sr⁺² and Zn⁺² and X is a member selected from the group consisting of chloride, bromide, iodide, citrate, gluconate, lactate, glycinate, glutamate, ascorbate, aspartate, nitrate, phosphate, hydrogenphosphate, dihydrogenphosphate, formate, maloneate, maleate, succinate, carbonate, bicarbonate, sulfate, and hydrogensulfate (particular salts being NaCl, CaCl₂ and ZnCl₂);

(e) 0-5% of a water soluble carbon based high refractive index (R.I.>1.4200) agent selected from the group consisting of glycerin, alanine, choline, lysine, diglycereth-7, triglycereth-7 citrate, glycereth-7 glycolate, glycereth-5 lactate, lauramidopropyl glycerin, glycereth-5 lactate, glycereth-7 glycolate, and glycereth-20 benzoate (Ethox GB-2) (for example, 0.5-3 weight % of a high refractive index agent selected from the group consisting of diglycereth-7 citrate, glycereth-7 glycolate, glycereth-5 lactate, lauramidopropyl glycerin, glycereth-5 lactate, glycereth-7 glycolate and glycereth-20 benzoate; and

(f) 0-10 weight % (particularly 0-4%) of an alcohol having 2-4 carbons (for example, 1-5% ethanol);

wherein:

(i) all amounts are based on the weight of the entire composition;

(ii) the composition is an emulsion having a viscosity greater than 150,000 centipoise (for example, in the range of 150,000-600,000 centipoise, more specifically, 200,000-350,000 centipoise);

(iii) the composition has a ratio of oil phase to water phase in the range of 10:90 to 24:76; and

(iv) the composition is free of glycine, elastomers, borate crosslinkers, soap gelling agents, insoluble suspended materials, and secondary water-soluble surfactants having an HLB value ≧9.

The invention also includes combinations of the subranges and subgroups described above.

Ideally the Betaine is incorporated during the synthesis of the salt so as to maximize the stabilizing effect this amino acid has on the zirconium salt species. Alternatively it can be post added along with additional active phase ingredients to form a Betaine stabilized active.

The refractive index of the active phase is matched to the oil phase (comprised of fragrance, surfactants, fragrance solubilizers silicones) by adding glycols, and optionally monovalent and/or divalent salts as well as monohydric alcohols such that the refractive index of the active phase differs from the refractive index of the oil phase by an amount of about 0.000 to 0.0040 units. This renders a clear product have an NTU (Nephelometric Turbidity Units) value in the range of between 50 and 250 and an R.I. in the range of 1.4025 to 1.4150. Most preferably the refractive index of the active phase is lower than that of the fragranced oil phase thereby minimizing the level of glycols. If added to the composition, the fragrance oils typically have refractive indices ranging from 1.4450-1.4850 and are used typically at levels ranging from 0.50-2.0 weight percent. Ionizable monovalent and divalent inorganic salts, antiperspirant salts, water and optional ingredients such as alcohol are optimized to provide a quick drying gel composition.

Refractive index measurements are made at a temperature of about 20-25 degrees C. using a Bausch and Lomb Abbe 3L Refractometer. Turbidity measurements as described herein are made with an Orbeco-Hellige #965 Direct-Reading Turbidimeter.

One particular embodiment is free of amino acids (Betaine hereby being excluded by definition from the class of amino acids.)

One embodiment of the invention is comprised of an oil phase composition such that the addition of the fragrance component (if fragrance is added to the composition) provides a refractive index in the range from about 1.4015 to about 1.4150; especially from about 1.4025 to about 1.4090.

One of the benefits of adding a non-siliconized organic fragrance solubilizer is to improve the solubility of the fragrance within the silicone (primarily cyclomethicones) based oil phase of the gel emulsion. The limited solubility of some fragrances in the cyclomethicone and linear polydialkylorganosiloxanes (if present) is easily determined by the turbidity of the aforementioned silicone(s) when approximately 10-30 weight % of fragrance is added to the silicone. To those skilled in the art, it is known that the addition of as little as 5-10 weight % (the amount depending on the fragrance) of the fragrance solubilizers previously described (e.g PPG-3 myristyl ether) to the silicone/fragrance mixture results in a clear solution.

The high viscosity gel compositions of the present invention include an antiperspirant active agent in an amount sufficient to have a deodorizing effect and/or in an amount sufficient to reduce the flow of perspiration when the composition is applied to a human. For the antiperspirant active used in the internal (also called “active”) phase various antiperspirant active materials that can be utilized according to the present invention provided that they are soluble at a suitable concentration in the active phase.

Antiperspirant actives can be incorporated into compositions according to the present invention in amounts in the range of 14-30 weight % (on an anhydrous solids basis), particularly 17-25 weight % of the total weight of the composition. Mixtures of actives can also be used. The amount used will depend on the formulation of the composition. At amounts at the higher end of the range a good antiperspirant effect can be expected. As noted above, the active is preferably included in the compositions of the invention by premixing the active with water and possibly small amount of propylene glycol.

Antiperspirant actives can be incorporated into compositions according to the present invention in amounts as described above. At lower levels the antiperspirant active material may not completely reduce the flow of perspiration, but will reduce malodor, for example, by acting as an antimicrobial material. At amounts of 15-25% by weight of the total weight of the composition (on an actives basis), an antiperspirant effect may be observed.

Particularly preferred are antiperspirant actives having the specific low metal to chloride ratio specified above and those described in U.S. Pat. No. 6,375,937 and patent application assigned to the same owners as this case (U.S. patent application Ser. No. 10/314,712 filed Dec. 9, 2002).

In one particular type of salt of interest, an aluminum zirconium tetrasalt or octasalt free of glycine are used wherein aluminum zirconium salt has a metal to chloride ratio in the range of 0.9 to 1.3:1 (especially in the range of 0.9 to 1.2:1 and, more particularly in the range of 0.9 to 1.1:1). For the tetrasalt the Al/Zr atomic ratio lies within the range of 3.2:1 to 4.1:1.0 and the Betaine:zirconium mole ratio lies within the range of 0.2-3.0:1 particularly 0.4-1.5:1).

One particular embodiment may be made with propylene glycol and an aluminum/zirconium tetrasalt so that the propylene glycol/Zr weight/weight ratio is in the range of 0.4-2.5.

One particular type of salt of interest is an aluminum chloride salt buffered by Betaine, wherein the salt has a metal to chloride ratio in the range of 0.9 to 1.3:1 (especially in the range of 0.9 to 1.2:1 and, more particularly in the range of 0.9 to 1.1:1). Examples of commercially available glycine-free, low M:Cl aluminum chloride salts include Westchlor 100 (from Westwood Chemicals). Other salts of interest include but are not limited to octasalts wherein the Al/Zr atomic ratio lies within the range of 6.2-10.0:1 and the Betaine:Zr mole ratio lies within the range of 0.2-3.0:1 (particularly 0.4-1.5:1). For the tetrasalt the Al/Zr atomic ratio lies within the range of 3.2:1.0 to 4.1:1.0 and the Betaine:Zr mole ratio lies within the range of 0.12-3.0:1.0 (particularly 0.4:1.5:1.0). Ideally, in the case of a salt that contains zirconium, the Betaine is incorporated during the synthesis of the salt so as to maximize the stabilizing effect this amino acid has (especially on the zirconium species). Alternatively it can be post added to a glycine free, salt along with additional active phase ingredients to form a Betaine stabilized active. Examples of commercially available glycine free low M:Cl ratio tetrasalts and octasalts include (but are not limited to) Rezal AZP 955 CPG and Rezal AZP 885 respectively (both from Reheis Chemical Company, Berkeley Heights, N.J.). A more detailed description of making such commercially available salts can be found for example, in U.S. Patent Application Publication US 2004/0001795A1 and U.S. application Ser. No. 10/625,038. Further examples of making this type of salt complexes are given in (but are not limited to) U.S. patent application Ser. Nos. 10/406,856 and 10/607,099 listed above. Also of interest are salts which include other amino acids such as alanine and lysine to further increase the refractive index of the Betaine-containing active complex.

The cyclomethicones used in this invention are one or more members selected from the group consisting of cyclic polydimethylsiloxanes such as those represented by Formula II: 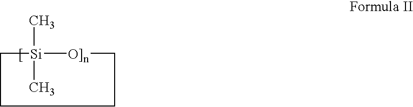
where n is an integer with a value of 4-6, particularly 5-6. These include a tetramer (D4), a pentamer (D5), and a hexamer (D6), and mixtures of any two or three of the forgoing. For example, DC-245 fluid and DC-345 from Dow Corning Corporation (Midland, Mich.) are types of cyclomethicones which can be used. It is to be noted that for purposes of the present invention cyclomethicones are not considered as silicone emollients.

Suitable silicone surfactants include silicone polyglucosides (for example, octyl dimethicone ethoxy glucoside) and silicone copolyols having an HLB value (hydrophilic lipophilic balance)≦8. The HLB value may be measured in a variety of ways such as described in conventional references or found listed in tables of data recording such values.

HLB values are a concept well known in the art, for example, as described in U.S. Pat. No. 6,682,749 and a variety of other references For purposes of this invention the test that should be used is the Three Dimensional HLB system (which is tailored for silicones) as described in O'Lenick, Jr., A. J., et al., Cosmetics & Toiletries, Vol. 112 :59-64 November 1997). For purposes of this invention it is intended that either the actual or theoretical HLB value may be used as the basis for selection.

A silicone copolyol (especially dimethicone copolyol) may be used in an amount of 0.6-0.9 weight % (actives basis), particularly 0.6-0.8.

In general, silicone copolyols useful in the present invention include copolyols of the following Formulae III and IV. Formula III materials may be represented by:
(R¹⁰)₃—SiO—[(R¹¹)₂—SiO]ₓ—[Si(R¹²)(R^{b}—O—(C₂H₄O)ₚ—(C₃H₆O)ₐ—R^{c})O]_{y}—Si—(R¹³)₃ Formula III
wherein each of R¹⁰, R¹¹, R¹² and R¹³ may be the same or different and each is independently selected from the group consisting of C1-C6 alkyl; R^{b} is the radical —CₘH₂ₘ; R^{c} is a terminating radical which can be hydrogen, an alkyl group of one to six carbon atoms, an acyl group such as an ester with a terminating alkyl group of 1-4 carbons, or an aryl group such as phenyl; m has a value of two to eight; p and s have values such that the oxyalkylene segment —(C₂H₄O)ₚ—(C₃H₆O)ₛ— has a molecular weight in the range of 200 to 5,000; the segment preferably having fifty to one hundred mole percent of oxyethylene units —(C₂H₄O)ₚ— and one to fifty mole percent of oxypropylene units —(C₃H₆O)ₛ—; x has a value of 8 to 400; and y has a value of 2 to 40. Preferably each of R¹⁰, R¹¹, R¹² and R¹³ is a methyl group; R^{c} is H; m is preferably three or four whereby the group R^{b} is most preferably the radical —(CH₂)₃—; and the values of p and s are such as to provide a molecular weight of the oxyalkylene segment —(C₂H₄O)ₚ—(C₃H₆O)ₛ— of between about 1,000 to 3,000. Most preferably p and s should each have a value of about 18 to 28.

A second siloxane polyether (copolyol) has the Formula IV:
(R¹⁰)₃—SiO—[(R¹¹)₂—SiO]ₓ—[Si(R¹²)(R^{b}—O—(C₂H₄O)ₚ—R^{c})O]_{y}—Si—(R¹³)₃ Formula IV
wherein p has a value of 6 to 16; x has a value of 6 to 100; and y has a value of 1 to 20 and the other moieties have the same definition as defined in Formula III.

It should be understood that in both Formulas I and II shown above, that the siloxane-oxyalkylene copolymers of the present invention may, in alternate embodiments, take the form of endblocked polyethers in which the linking group R^{b}, the oxyalkylene segments, and the terminating radical R^{c} occupy positions bonded to the ends of the siloxane chain, rather than being bonded to a silicon atom in the siloxane chain. Thus, one or more of the R¹⁰, R¹¹, R¹² and R¹³ substitutents which are attached to the two terminal silicon atoms at the end of the siloxane chain can be substituted with the segment —R^{b}—O—(C₂H₄O)ₚ—(C₃H₆O)ₛ—R^{c} or with the segment —R^{b}—O—(C₂H₄O)ₚ—R^{c}. In some instances, it may be desirable to provide the segment —R^{b}—O—(C₂H₄O)ₚ—(C₃H₆O)ₛ—R^{c} or the segment —R^{b}—O—(C₂H₄O)ₚ—R^{c} at locations which are in the siloxane chain as well as at locations at one or both of the siloxane chain ends.

Particular examples of suitable dimethicone copolyols are available either commercially or experimentally from a variety of suppliers including Dow Corning Corporation, Midland, Mich.; General Electric Company, Waterford, N.Y.; Witco Corp., Greenwich, Conn.; and Goldschmidt Chemical Corporation, Hopewell, Va. Examples of specific products include DOW CORNING® 5225C from Dow Corning which is a 10% dimethicone copolyol in cyclomethicone; DOW CORNING® 2-5185C which is a 45-49% dimethicone copolyol in cyclomethicone; SILWET L-7622 from Witco; ABIL EM97 from Goldschmidt which is a 85% dimethicone copolyol in D5 cyclomethicone; and various dimethicone copolyols available either commercially or in the literature.

It should also be noted that various concentrations of the dimethicone copolyols in cyclomethicone can be used. While a concentration of 10% in cyclomethicone is frequently seen commercially, other concentrations can be made by stripping off the cyclomethicone or adding additional cyclomethicone. The higher concentration materials such as DOW CORNING® 2-5185 material is of particular interest.

In one particular embodiment 3-9 weight % (particularly 5-8%) of a 10% silicone copolyol such as dimethicone copolyol in cyclomethicone mixture may be used, wherein the amount of mixture added is selected so that the level of silicone copolyol in the cosmetic composition is in the range of 0.6-0.9% particularly 0.6-0.8%) (for example, 6.5% of a 10% dimethicone copolyol in cyclomethicone mixture).

Emollient selection is limited to those which have a relatively low refractive index (R.I.<1.4200) in order to reduce the refractive index of the oil phase thereby making it easier to match the refractive index of the internal active phase. Emollients are a known class of materials in this art, imparting a soothing effect to the skin. They are ingredients which help to maintain the soft, smooth and pliable appearance of the skin. Emollients are also known to reduce whitening on the skin and/or improve aesthetics. Examples of suitable emollients which meet the low refractive index requirement are limited to silicone-based structures which are absent of phenyl groups.

Preferably linear silicones, are selected from the group consisting of polydialkylsiloxanes represented by the formulae:
(1) (R¹⁰)₃SiO(Si(R¹¹)₂O)ₓSi(R¹²)₃ where R¹⁰, R¹¹ and R¹² can be the same or different and are each independently selected from the group consisting of C1-C10 alkyl (with particular examples being polydimethylsiloxanes (dimethicones), especially low viscosity dimethicones (viscosity<100 cps) (with the R groups and x values being selected to maintain the RI limits);
(2) silanols and/or dimethiconols in which one or two of the alkyl (such as methyl) groups of the foregoing formula may be replaced with a hydroxyl group and is represented by formulae:

HO(R¹⁴)₂SiO(Si(R¹⁵)₂O)ₓSi(R¹⁶)₂OH and R17(R¹⁴)₂SiO(Si(R¹⁵)₂O)ₓSi(R¹⁶)₂OH where R¹⁴, R¹⁵ and R¹⁶ and R¹⁷ can be the same or different and are each independently selected from the group consisting of C1-C10 alkyl (with a particular example being methyl) (with the R groups and x values being selected to maintain the RI limits).

Particular examples of suitable silicone emollients include members of the group consisting of linear silicones (both volatile and non-volatile) such as linear dimethicones, particularly dimethicones having a viscosity in the range of 0.5-300,000 centistokes, indicated in Formula V, with t values being selected to maintain the viscosity limits. Most suitable are low viscosity dimethicones (viscosity<1,000 centistokes (e.g. DC 200) including hexamethyl disiloxane.

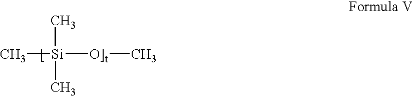

Further examples of suitable silicone emollients include, dimethiconols (such as but not limited to Dow Corning® DC1501), Dow Corning 2501 cosmetic wax (dimethicone copolyol) dimethiconol behenate, C₃₀₋₄₅ alkyl methicone, stearoxytrimethylsilane, and stearyl dimethicone) as well as silanol DC 9023 also from Dow Corning).

The low refractive index emollient or emollient mixture or blend thereof incorporated in compositions according to the present invention can, illustratively, be included in amounts of 0-1 weight % inclusive, preferably 0.5-1%.

The oil phase according to the present invention is, desirably, a silicone oil phase, so as to provide a water-in-silicone oil emulsion. The total of oil phase and siloxane surface-active agent preferably makes up from about 10% to about 24% by weight, of the total weight of the composition. This surface-active agent is an emulsifier which, when properly mixed with the aqueous phase components, and oil phase components, yields a water-in-oil emulsion. The oil phase is desirably a blend of liquids, but does not contain any significant amount of non-volatiles (that is, less than 1.0 weight % of any material having a flash point greater than 100 degrees C.). Moreover, the nonvolatile silicone based emollients described in this composition all have a refractive index lower than 1.420.

The refractive index of the oil phase (including fragrance) is determined, and, if necessary, adjusted to be in the range of 1.4015 to about 1.4150; especially from about 1.4025 to about 1.4090 and the refractive index of the aqueous phase is determined and adjusted (if necessary) to differ from the refractive index of the oil phase by less than 0.0050, preferable less than 0.0030 units. The aqueous phase is then mixed with the oil phase (for example, the aqueous phase is slowly added to the oil phase with turbulent agitation), and then additional additives, or other active ingredients, are added with mixing. Alternatively the fragrance can be added after the active phase is added to the oil phase. In this instance the refractive index of the oil phase (absent of fragrance) and aqueous phase should differ less than 0.0020. The resulting emulsion is then passed through, for example, a colloid mill or other high shear emulsifier so as to provide a viscous gel, the gel then being transferred to a suitable applicator or container for use by the consumer. Desirably, according to the present invention the aqueous phase further includes a glycol mixture consisting of propylene glycol and 2-methyl propanediol and/or dipropylene glycol providing advantages in the final product as discussed previously.

A particular example of an alkoxylated, alkyl substituted siloxane surface active agent is preferably, but not limited to, a dimethicone copolyol. An illustrative alkoxylated silicone-containing surfactant utilizable according to the present invention is cetyl dimethicone copolyol, referred to in U.S. Pat. No. 5,162,378 to Guthauser. Illustratively, the alkoxylated, alkyl substituted siloxane surface active agent is included in the composition in an amount of 6.0% to 9.0% by weight, of the total weight of the composition. Another example of a suitable surfactant is octyl dimethicone ethoxy glucoside (from Wacker-Belsil, Adrian, Mich.).

A specific cyclomethicone-dimethicone copolyol fluid which can be utilized to provide the alkoxylated silicone-containing surface-active agent is a mixture of cyclomethicone and dimethicone copolyol designated as DC 5225C from Dow Corning Corporation. This is a polyether substituted silicone of cyclomethicone and dimethicone copolyol (refractive index (RI)=1.3994) at about 20-25 degrees C. This DC 5225C, which is an emulsifying agent, is useful for preparing stable water-in-oil emulsions where a silicone makes up a large portion of the oil phase, and is a dispersion of a silicone surfactant (dimethicone copolyol) (10% by wt.) in cyclomethicone (Dow Corning 245) (90% by weight).

The total mixture of cyclomethicone and dimethicone copolyol fluid is present in the composition, illustratively, in an amount of from about 12.5% to about 23.9% by weight, of the total weight of the composition.

According to another aspect of the present invention, the aqueous phase of the clear cosmetic gel composition further includes a glycol system in low amount (3.5-10 weight % preferably 4-8%) comprising propylene glycol and, preferably, at least one other glycol or polyglycol to help increase the refractive index of the active phase so as to match it within 0.0000-0.0040 units (preferably 0.0010-0.0030 units) of the fragranced oil phase. Illustratively, tripropylene glycol can be utilized as the additional polypropylene glycol. According to this aspect of the present invention, propylene glycol can be used in combination with the glycols up to a limited amount. Incorporation of glycols, particularly MP-diol (2-methyl 1,3 propanediol) and dipropylene glycol in the gel composition also improves cosmetic properties, including a reduction of tack and a decrease in the whitening and in the residue after application of the composition. Moreover, compositions incorporating polypropylene glycol, particularly, tripropylene glycol, have improved mildness (that is, reduced skin irritation potential) relative to commercially available products. The glycol or polyglycol is selected from the group consisting of ethylene glycol, propylene glycol, 1,2 propanediol, 2-methyl propanediol, diethylene glycol, triethylene glycol, tetraethylene glycol, dipropylene glycol, tripropylene glycol, methyl propanediol, 1,6-hexanediol, 1,3 butanediol, 1,4 butanediol, PEG-4 through PEG-600, PPG-9 through PPG-34, neopentyl glycol, trimethylpropanediol, 2,2 dimethyl-1,3 propanediol, 2,2,4,4-tetramethyl-1,3-cyclobutane-diol and mixtures thereof. More particular examples of the glycols which can be used with the propylene glycol are one or more members of the group consisting of dipropylene glycol, 2-methyl-1,3 propanediol, methyl propylene glycol, low molecular weight (less than 600) polyethylene glycol and mixtures of any of the foregoing in which the amount of propylene glycol does not exceed 7.5%.

In one embodiment, for example, the aqueous phase further includes a glycol mixture consisting of propylene glycol and 2-methyl propanediol and/or dipropylene glycol providing advantages in the final product as discussed previously.

Clear gel compositions of the invention may further include at least one ionizable inorganic salt which helps to increase the refractive index of the active phase and optimize the glycol level. Higher levels of glycols, although good for reducing tackiness tend to increase dry-down time. These ionizable salts are of the form MₐX_{b} where a=1, or 2 and b=1 or 2; M is a member selected from the group consisting of Na⁺¹, Li⁺¹, K⁺¹, Mg⁺², Ca⁺², Sr⁺² and Zn⁺² and X is a member selected from the group consisting of chloride, bromide, iodide, citrate, gluconate, lactate, glycinate, glutamate, ascorbate, aspartate, nitrate, phosphate, hydrogenphosphate, dihydrogenphosphate, formate, maloneate, maleate, succinate, carbonate, bicarbonate, sulfate, hydrogensulfate. Salts of particular utility are NaCl and ZnCl₂. As will be appreciated by those skilled in the art, while it may be possible under certain circumstances to add a salt directly to a portion of the mixture during manufacturing, it is preferred to add the salt as a mixture or solution of the salt in a carrier or solvent, particularly water. Of course various concentrations of the salt premix can be made such as in the range of 1-40%.

The present invention also includes methods of forming high viscosity cosmetic gel compositions described herein. In such methods an aqueous phase comprising water and the antiperspirant active is formed separately from the oil phase containing an alkoxylated, alkyl substituted siloxane surface active agent, an organic fragrance solubilizer and cyclomethicone and optional fragrance. The two phases are then combined and homogenized to achieve the desired viscosity.

For one embodiment the active phase is a water phase containing 17-30 weight % (anhydrous) of the low metal:chloride Betaine-containing antiperspirant active, 3.5-10 weight % of the water-soluble glycol system and optionally 0-4% the monovalent or divalent ionizable water-soluble salt. Illustratively, free water is included in the composition in the range of 30-70 weight % based on the total weight of the composition. The water phase can also include, for example, (along with the antiperspirant active, water-soluble glycol system and ionizable divalent or monovalent salt) 1-5 weight % of a water-soluble carbon-based, high refractive index agent such as glycerin These compositions of the present invention may be prepared by a batch process, or a continuous or semi-continuous process, and the processes yield compositions which are stable, highly efficacious and possess excellent aesthetic qualities.

The compositions according to the present invention are used as conventional cosmetic gel compositions. For example, where the composition according to the present invention is a clear antiperspirant soft gel composition, packaged in a dispensing container having a top surface with slots or pores appropriate for the viscosity range of this product, the gel is extruded from the dispensing container through the slots or pores and applied to the skin (for example, in axillary regions of the human body) by rubbing the soft gel material extruded through the top surface of the container on the skin in the axillary region.

As a further aspect of the present invention, the dispensing container can be clear and can be tinted so as to for example, fit to the fragrance hedonics. The composition has reduced tack, quick dry down, a cool sensation, and a silky feel and imparts much less or no white residue on dry down compared to commercially available products. Moreover, compositions of the present invention incorporating a polypropylene glycol component have improved mildness (have reduced skin irritation potential) as compared to commercially available products, and have improved cosmetic properties (including reduced tackiness) and reduced white residue upon application. The gel emulsions according to the present invention are stable and optically clear are cosmetically elegant, and are capable of being delivered from a suitable applicator package.

Throughout the present specification, the antiperspirant active materials, when utilized in an antiperspirant effective amount in the composition, act to reduce body malodor by reducing production of perspiration; however, these antiperspirant active materials can also have a deodorant function, e.g., as an antimicrobial agent. The deodorant active materials do not substantially reduce the production of perspiration, but reduce malodor in other ways, e.g., as fragrances masking the malodor or reducing the malodor intensity, as odor absorbents, as antimicrobial agents, as agents chemically reacted with malodorous materials, etc.

The amount of active component that can be used will vary with the particular active ingredient incorporate. The product comprises antiperspirant active materials in amounts sufficient to combat body malodor either as a deodorant or as an antiperspirant when applied to the axillary regions of the body. As a general rule, an antiperspirant product should contain an active antiperspirant material in an amount anywhere from about 9% to about 25% by weight, of the total weight of the composition. However, for the present invention it has been found that a minimum of at least 14 weight % of antiperspirant salt must be used to obtain the clarity desired. The active antiperspirant material utilized in the compositions of the present invention can be pre-dissolved in water or in another solvent (for example, in propylene glycol), and may be buffered or unbuffered. Preferably, the antiperspirant materials are present in solution in a solvent.

Where a deodorant active material is utilized other than lower amounts of an antiperspirant active, any deodorant active material, which can be dissolved in the oil phase, can be utilized in an amount sufficient to have a deodorant effect. Illustratively, the deodorant active material can be 2,4,4′-trichloro-2′-hydroxy diphenyl ether (triclosan), and/or benzethonium chloride and/or octoxyglycerin (Sensiva® SC 50). Where the deodorant ingredient is used in place of the antiperspirant active ingredient, a deodorant gel composition (rather than an antiperspirant gel composition) would be provided.

With respect to embodiments comprising an alpha, beta-unsaturated ester malodor counteracting material, a number of examples can be described. These include clear gels wherein:

(a) the fragrance level is less than 0.1 weight % and the alpha, beta-unsaturated ester malodor counteracting material is between 0.1 and 0.4 weight %;

(b) the fragrance level is between 0.1 and 2.0% and the alpha, beta-unsaturated ester malodor counteracting material is between 0.1 and 0.4%;

(c) the gel comprises an alpha, beta-unsaturated ester malodor counteracting material comprising a combination of two or more members selected from the following groups:
(1) 3-phenyl-2-propenoic acid alkyl esters wherein R¹ is a substitutent on the benzene ring and is an alkyl, an alkoxy, an aryl, or a substituted aryl, preferably R¹ is H, a C1 to C8 alkyl, a C1 to C8 alkoxy, or an aryl; and R² is a subsistent group replacing the carboxylic acid hydrogen to form the ester where R² has greater than 6 carbon atoms, an aryl, or a substituted aryl group, preferably, R² is a C6 to C12 alkyl or is a benzyl group; and
(2) an ester of fumaric or maleic acid having linear ester carbon chains from 3-9 carbons, for example dihexyl fumarate;

(d) a gel comprising:
(1) an alpha, beta-unsaturated ester malodor counteracting material comprising an e-phenyl propenoic acid ester selected from the group consisting of octyl methoxy cinnamate, phenylethyl cinnamate, benzyl cinnamate; and
(2) an aliphatic unsaturated ester selected to be dihexyl fumarate;

(e) a gel comprising a combination of two or more members selected from the group consisting of:
(1) 3-phenyl-2-propenoic acid alkyl esters wherein R¹ is a substitutent on the benzene ring and is an alkyl, an alkoxy, an aryl, or a substituted aryl, preferably R¹ is H, a C1 to C8 alkyl, a C1 to C8 alkoxy, or an aryl; and R² is a substitutent group replacing the carboxylic acid hydrogen to form the ester where R² is a C6 to C12 alkyl or is a benzyl group; and
(2) an ester of fumaric or maleic acid having linear ester carbon chains from 3-9 carbons, for example dihexyl fumarate; and

(f) a gel wherein the alpha, beta-unsaturated ester malodor counteracting material is a mixture of:
(1) an e-phenyl propenoic acid ester selected from the group consisting of octyl methoxy cinnamate, phenylethyl cinnamate, benzyl cinnamate; and
(2) dihexyl fumarate.

Throughout the present specification, where compositions are described as including or comprising specific components or materials, it is contemplated by the inventors that the compositions of the present invention also consist essentially of, or consist of, the recited components or materials. Accordingly, throughout the present disclosure any described composition of the present invention can consist essentially of, or consist of, the recited components or materials.

A desired feature of the present invention is that a clear cosmetic gel composition (e.g., clear deodorant or antiperspirant gel composition) can be provided. The term clear (that is clarity), according to the present invention, is intended to connote its usual dictionary definition; thus, a clear, e.g., cosmetic gel composition of the present invention allows ready viewing of objects behind it. By contrast, a translucent composition allows light to pass through, but causes the light to be so scattered that it will be impossible to see objects behind the translucent composition. Optical clarity of compositions of the present invention can be measured using a turbidimeter as described above, and desirably lies between 50 and 250 NTU measured at room temperature (20°-25° C.).

Moreover, the clear cosmetic gel composition of the present invention, which is in the form of a macro-emulsion as contrasted to a micro-emulsion, does not need to contain wax or gelling agents such as soaps, cellulosic materials or alginates. Furthermore, the composition according to the present invention does not require polydimethylcyclosiloxane, although the present compositions may contain this material.

Several particular embodiments include those comprising one or more of the following: 1-4 weight % of ethanol or propanol; 9-20 weight % of the one or more cyclomethicones; 0.6-0.8 weight % of the silicone surfactant; 0.5-2 weight % of the fragrance solubilizer; 45-65 weight % water; and a glycol system comprising 3-7 weight % of propylene glycol in combination with either 2-methyl propane diol or dipropylene glycol.

Various materials incorporated in the water-based phase and in the oil-based phase, and their refractive indices (as measured using the Bausch and Lomb Abbe 3L Refractometer) are set forth in the following particular formulations:
6.0-9.0 weight % dimethicone copolyol/cyclomethicone (10%) (for example, Dow Corning 5225C);
3.9-17.9 weight % (preferably 8-15 weight percent) cyclomethicone (D4, D5, D6 or mixtures thereof);
0.1-3.0 weight % PPG-3 myristyl ether;
17-25 weight % antiperspirant active (for example, Al—Zr tetrachlorohydrex such as Rezal AZP 955 CPG, or Al—Zr octasalt such as Rezal AZP 885 to which Betaine has been added so that the molar ratio of Betaine/Zr is in the range of 0.2-2.0 or aluminum dichlorohydrate (such as Westchlor 100, 36.1%) to which Betaine has been added so that the molar ratio of Betaine/Al is in the range of 0.05-1.0:1;
30-70 weight % water;
0.2 to 4.0 weight % of an ionizable salt or combinations of ionizable salts of the form MₐX_{b} where a=1 or 2; b=1 or 2; M is a member selected form the group consisting of Na⁺¹, Li⁺¹, K⁺¹, Mg⁺², Sr⁺² and Zn⁺², Ca⁺² and X is a member selected from the group consisting of chloride, bromide, iodide, citrate, gluconate, lactate, glycinate, glutamate, ascorbate, aspartate, nitrate, phosphate, hydrogenphosphate, dihydrogenphosphate, formate, malonate, maleate, succinate, carbonate, bicarbonate, sulfate and hydrogensulfate (preferred salts are ZnCl₂, CaCl₂ and NaCl or combinations thereof);
3.5-8% weight % of the glycol system as described above in the definition of the invention;
0-1 weight % of a low refractive index emollients (RI<1.4200) such as dimethiconol, dimethicones, silanol and combinations thereof;
0-5 weight % fragrance or odor masking component;
0.0-0.45 weight % of an odor neutralizing alpha, beta, unsaturated ester or mixtures thereof;
0-10 weight % of a water soluble non-glycol organic solvent selected from the group consisting of 2-4 carbon chain alcohols (for example ethanol);
0-5 weight % of a water-soluble carbon based high refractive index agent such as additional Betaine which is not part of the original salt, alanine, choline, lysine, glycerin, Diglycereth-7, Triglycereth-7 citrate, glycereth-7 glycolate, glycereth-5 lactate, lauramidopropyl glycerin, glycereth-5 lactate, Glycereth-7 glycolate, and glycereth-20 benzoate;
   wherein the composition has a phase ratio in the range of 10:90-24:76 of oil to water and a viscosity is in the range of 150,000-600-000 centipoise.

### EXAMPLES

The following Examples are offered as illustrative of the invention and are not to be construed as limitations thereon. In the Examples and elsewhere in the description of the invention, chemical symbols and terminology have their usual and customary meanings. In the Examples as elsewhere in this application values for n, m, etc. in formulas, molecular weights and degree of ethoxylation or propoxylation are averages. Temperatures are in degrees C. unless otherwise indicated. The amounts of the components are in weight percents based on the standard described; if no other standard is described then the total weight of the composition is to be inferred. Various names of chemical components include those listed in the CTFA International Cosmetic Ingredient Dictionary (Cosmetics, Toiletry and Fragrance Association, Inc., 7^{th} ed. 1997). Refractive Indices (“RI”) are determined at a temperature in the range of 20-25 degrees C. Some illustrative methods of preparing active phases containing Betaine stabilized aluminum zirconium tetrasalt and octasalts in which the Betaine is added during the synthesis of the AP salt are given below in Examples 1 and 2. The internal phase may also be referred to as the active phase or the water phase. As will be understood by those skilled in the art, the common and conventional usage of ratio is on a molar basis, except for Al/Zr ratios which are on an atomic basis and propylene glycol/Zr ratios which are on a weight percent basis.

### Example 1

A Betaine-stabilized antiperspirant salt solution may be made by dissolving 19.26 g of ZrOCl₂.8H₂O in 49.6 g of water and then adding 8.39 g Betaine anhydrous. After everything is dissolved, an aluminum chlorohydrate (“ACH”) powder (22.65 g of Chlorhydrol from Reheis Chemical Co., Berkeley Heights, N.J.) into the solution with additional deionized (“DI”) water so that the total weight of the solution is 100 g. The solution is shaken or stirred to make sure the solution is clear. Optionally, the solution can be spray dried or freeze-dried to make a powder sample. This 30% salt solution (anhydrous basis) has the following composition:

| Al/Zr = 3.5 | Metal/Cl = 1.2 | Betaine/Zr = 1.2 | |
|---|---|---|---|
| Al: | 5.64% | 0.00209 | mole |
| Zr: | 5.45% | 0.000597 | mole |
| Cl: | 7.95% | 0.00224 | mole |
| Betaine | 8.39% | 0.000716 | mole |

To this Betaine stabilized salt solution various other active phase components such as the glycols and additional water are added to make up the active phase. The amounts of the various active phase components along with the optional active phase ingredients such as monovalent and/or divalent salts and short chain (2-4 carbons) alcohols are optimized for skin feel as well as for RI index so as provide a clear, aesthetically pleasing emulsion gel.

### Example 2

A Betaine-stabilized AP salt solution may be made by dissolving 18.15 g of ZrOCO₃x8H₂O in 5.95 g of concentrated HCl (37%) and 20 g of water (such as deionized (“DI”) water). After a clear solution is formed, 9.17 g of Betaine hydrochloride is added and stirred until dissolved. Subsequently, 22.65 g of ACH powder (Chlorhydrol from Reheis Chemical Co., Berkeley Heights, N.J.) is added into the solution with additional DI water so that the total weight of the solution is 100 g. The solution is shaken or stirred to make sure the solution is clear. This 30% salt solution (anhydrous basis) has the following composition:

| Al/Zr = 3.5 | M/Cl = 1.2 | Betaine/Zr = 1.0 | |
|---|---|---|---|
| Al: | 5.64% | 0.00209 | mole |
| Zr: | 5.45% | 0.000597 | mole |
| Cl: | 7.95% | 0.00224 | mole |
| Betaine | 7.00% | 0.000597 | mole |

As in Example 1, this active is mixed with additional active phase components to render the active phase.

Alternatively the aluminum/zirconium tetra salt can be made separately according to, for example, patent application Ser. No. 10/185,299. The Betaine along with the other active phase ingredients is then post added to the active solution by mixing the components together at room temperature to render the active phase. This method of preparing the active phase results in a Betaine complexed active salt.

### Examples 3-28

Examples 3-28 shown in Tables A through C describe some illustrative methods which can be used (but do not limit) to prepare gel emulsions in which the sample sizes are about 500 grams. The oil phase is prepared by weighing and combining silicone copolyol, cyclomethicone, fragrance and fragrance solubilizer along with any optional oil phase components in a beaker. The mixture is stirred at 400-600 rpm using a Lightnin' Mixer Model LI003. After the mixture becomes visually homogeneous, the active phase (see above description) are added to the oil phase while mixing. The entire mixture is mixed for 15 minutes. The mixture is then homogenized for 2-4 minutes at a reading of 50-70 on Powerstat Variable Transformer (Superior Electric Co., Bristol, Conn.) using a homogenizer from Greerco Corp. (Hudson, N.H.). Examples 8 and 9 are made with the same composition as Examples 6 and 7, respectively but additionally include an odor neutralizing alpha, beta-unsaturated ester as described above.

TABLE A

| Ingredient (weight %) | Ex 3 | Ex 4 | Ex 5 | Ex 6 | Ex 7 | Ex 8 | Ex 9 |
|---|---|---|---|---|---|---|---|
| Cyclomethicone DC | 11.50 | 11.50 | 11.00 | 11.00 | 11.00 | 11.00 | 11.00 |
| 245 | | | | | | | |
| Dimethicone copolyol/ | 6.5 | 6.5 | 6.50 | 6.50 | 6.50 | 6.50 | 6.50 |
| Cyclomethicone (Dow | | | | | | | |
| Corning 5225C, 10%) | | | | | | | |
| PPG-3 myristyl ether | 1.00 | 1.00 | 1.50 | 1.50 | 1.50 | 1.50 | 1.50 |
| Fragrance | 1.0 | 1.0 | 1.00 | 1.00 | 1.00 | 0.9 | 0.6 |
| Malodor counteracting | 0.00 | 0.00 | 0.00 | 0.00 | 0.00 | 0.10 | 0.40 |
| alpha, beta unsaturated | | | | | | | |
| ester mixture | | | | | | | |
| Active A** | 55.0 | 55.0 | 55.00 | 0.00 | 0.00 | 0.00 | 0.00 |
| Active B*** | 0.0 | 0.0 | 0.00 | 0.00 | 65.00 | 0.00 | 65.00 |
| Active B**** | 0.0 | 0.0 | 0.00 | 66.60 | 0.00 | 66.60 | 0.00 |
| Westchlor 100***** | 0.0 | 0.0 | 0.00 | 0.00 | 0.00 | 0.00 | 0.00 |
| Water | 13.2 | 12.2 | 11.6 | 0.00 | 1.60 | 0.00 | 1.60 |
| Tripropylene glycol | 4.0 | 6.0 | 0.0 | 0.00 | 5.00 | 0.00 | 5.00 |
| MP Diol 2-methyl 1,3 | 0.0 | 0.0 | 0.0 | 0.00 | 0.00 | 0.00 | 0.00 |
| propanediol | | | | | | | |
| Dipropylene glycol | 0.0 | 0.0 | 5.0 | 0.00 | 0.00 | 0.00 | 0.00 |
| 1,3 propanediol | 0.0 | 0.0 | 0.0 | 5.00 | 0.00 | 5.00 | 0.00 |
| Zinc chloride | 0.0 | 0.0 | 2.6 | 2.60 | 2.60 | 2.60 | 2.60 |
| (70.5 w/w % aqueous | | | | | | | |
| solution) | | | | | | | |
| NaCl | 1.0 | 0.0 | 0.0 | 0.00 | 0.00 | 0.00 | 0.00 |
| Ethanol | 3.5 | 3.5 | 3.5 | 3.50 | 3.50 | 3.50 | 3.50 |
| Betaine | 3.0 | 3.0 | 2.0 | 2.00 | 2.00 | 2.00 | 2.00 |
| Propylene glycol | 0.3 | 0.3 | 0.3 | 0.30 | 0.30 | 0.30 | 0.30 |

**Al 885 Aluminum Zirconium octasalt 35% in water solution from Reheis Chem. Co
***Al dichlorohydrate 30% in water solution from Reheis Chem. Co.
****Al dichlorohydrate 29% in water solution from Reheis Chem. Co.

TABLE B

| Ingredient (weight %) | Ex. 10 | Ex. 11 | Ex.12 | Ex 13 | Ex 14 | Ex. 15 | Ex 16 |
|---|---|---|---|---|---|---|---|
| Cyclomethicone | 10.7 | 11.00 | 11.00 | 8.50 | 11.0 | 11.0 | 11.0 |
| (DC 245) | | | | | | | |
| Dimethicone copolyol/ | 6.50 | 6.50 | 6.50 | 9.00 | 6.50 | 6.50 | 6.50 |
| Cyclomethicone | | | | | | | |
| (Dow Corning 5225C, | | | | | | | |
| 10%) | | | | | | | |
| PPG-3 myristyl ether | 1.80 | 1.50 | 1.50 | 1.50 | 1.50 | 1.50 | 1.50 |
| Fragrance | 1.00 | 1.00 | 1.00 | 1.00 | 1.00 | 1.00 | 1.00 |
| Active A* | 56.50 | 54.00 | 54.00 | 54.00 | 0.00 | 0.00 | 0.00 |
| Active A** | 0.00 | 0.00 | 0.00 | 0.00 | 52.60 | 52.6 | 52.2 |
| Active B*** | 0.00 | 0.00 | 0.00 | 0.00 | 0.00 | 0.0 | 0.00 |
| Active B**** | 0.00 | 0.00 | 0.00 | 0.00 | 0.00 | 0.0 | 0.00 |
| Westchlor 100***** | 0.00 | 0.00 | 0.00 | 0.00 | 0.00 | 0.0 | 0.00 |
| Water | 13.70 | 13.40 | 14.40 | 12.8 | 13.8 | 14.90 | 18.2 |
| Tripropylene glycol | 0.00 | 4.5 | 3.5 | 4.70 | 3.6 | 3.6 | 4.5 |
| MP Diol | 0.00 | 0.00 | 0.00 | 0.00 | 0.00 | 0.0 | 0.00 |
| Dipropylene glycol | 1.70 | 0.00 | 0.00 | 0.00 | 0.00 | 5.5 | 0.00 |
| 1,3 propanediol | 0.00 | 0.00 | 0.00 | 0.00 | 0.00 | 0.0 | 0.00 |
| Zinc chloride (70.5 | 2.60 | 2.60 | 2.60 | 1.40 | 0.00 | 1.25 | 0.00 |
| w/w % aqueous solution) | | | | | | | |
| NaCl | 0.00 | 0.00 | 0.00 | 0.00 | 0.00 | 0.9 | 0.9 |
| Ethanol | 3.50 | 3.50 | 3.50 | 3.50 | 3.50 | 3.50 | 0.0 |
| Betaine | 3.00 | 2.00 | 2.00 | 3.6 | 6.50 | 2.25 | 4.2 |
| Propylene glycol | 0.00 | 0.00 | 0.00 | 0.00 | 0.00 | 0.0 | 0.00 |

*Al—Zr tetrachlorohydrex complex (AZP 955), 35% in water solution containing 5% PG from Reheis Chem. Co.
**Al—Zr tetrachlorohydrex complex (AZP 955), 36% in water solution containing 5% PG from Reheis Chem. Co.
****Aluminum dichlorohydrate complex (36% in water from Westwood Chemicals, Middletown, N.Y.)

TABLE C

| Ingredient (weight %) | Ex. 17 | Ex. 18 | Ex. 19 | Ex. 20 | Ex. 21 | Ex. 22 | Ex. 23 | Ex. 24 | Ex. 25 | Ex. 26 | Ex. 27 | Ex. 28 |
|---|---|---|---|---|---|---|---|---|---|---|---|---|
| Cyclomethicone | 11.5 | 11.0 | 11.0 | 11.0 | 11.0 | 11.0 | 11.0 | 11.0 | 11.0 | 11.0 | 11.0 | 11.0 |
| (DC 245) | | | | | | | | | | | | |
| Dimethicone | 6.5 | 6.5 | 6.5 | 6.5 | 6.5 | 6.5 | 6.5 | 6.5 | 6.5 | 6.5 | 6.5 | 6.5 |
| copolyol/ | | | | | | | | | | | | |
| Cyclomethicone | | | | | | | | | | | | |
| (Dow Corning | | | | | | | | | | | | |
| 5225C, 10%) | | | | | | | | | | | | |
| PPG-3 myristyl ether | 1.0 | 1.5 | 1.5 | 1.5 | 1.5 | 1.5 | 1.5 | 1.5 | 1.5 | 1.5 | 1.5 | 1.5 |
| Fragrance | 1.0 | 1.0 | 1.0 | 1.0 | 1.0 | 1.0 | 1.0 | 1.0 | 1.0 | 1.0 | 1.0 | 1.0 |
| Active A** | 55.5 | 54.0 | 52.0 | 52.6 | 52.6 | 52.5 | 52.0 | 52.0 | 52.0 | 52.0 | 52.6 | 52.2 |
| MP Diol | 0.0 | 0.0 | 0.0 | 0.0 | 0.0 | 0.0 | 0.0 | 0.0 | 0.0 | 0.0 | 0.0 | 0.0 |
| Dipropylene glycol | 0.0 | 0.0 | 0.0 | 0.0 | 0.0 | 0.0 | 0.0 | 0.0 | 0.0 | 0.0 | 0.0 | 0.0 |
| Tripropylene glycol | 3.7 | 5.0 | 4.2 | 3.6 | 3.6 | 4.7 | 5.5 | 5.5 | 4.0 | 5.5 | 3.5 | 4.5 |
| 1,3 Propanediol | 0.0 | 0.0 | 0.0 | 0.0 | 0.0 | 0.0 | 0.0 | 0.0 | 0.0 | 0.0 | 0.0 | 0.0 |
| Betaine | 2.2 | 2.2 | 3.2 | 6.5 | 5.4 | 3.0 | 2.6 | 3.2 | 3.7 | 4.2 | 3.7 | 3.7 |
| Water | 12.5 | 13.5 | 15.6 | 13.8 | 14.9 | 15.5 | 15. | 14.9 | 15.6 | 14.3 | 14.95 | 15.2 |
| Zinc chloride sol. | 2.6 | 1.8 | 0.0 | 0.0 | 0.0 | 0.0 | 1.2 | 0.9 | 1.2 | 0.5 | 1.5 | 0.9 |
| (70.5% w/w% aqueous | | | | | | | | | | | | |
| solution) | | | | | | | | | | | | |
| NaCl (granular) | 0.0 | 0.0 | 1.5 | 0.0 | 0.0 | 0.8 | 0.0 | 0.0 | 0.0 | 0.0 | 0.0 | 0.0 |
| Ethanol | 3.5 | 3.5 | 3.5 | 3.5 | 3.5 | 3.5 | 3.5 | 3.5 | 3.5 | 3.5 | 3.75 | 3.5 |
| Total | | | | | | | | | | | | |

**Al+113 Zr tetrachlorohydrex complex (AZP 955), 36% in water solution containing 5% PG from Reheis Chem. Co.

1. An elastomer-free, suspension-free, water-in-oil emulsion which is a clear gel having a clarity of 50-250 NTU at 21.0 degrees C.; a viscosity >150,000 centipoise; and an overall level of silicone emollients ≦3 weight %, wherein the clear gel comprises:
(a) an internal phase comprising:
(i) 14-30 weight % of a glycine-free antiperspirant active with a low metal to chloride ratio in the range of 0.9-1.3:1 and stabilized by Betaine;
(ii) a high water content such that the water level of the entire composition is >30 weight % water based on the total gel composition; and
(iii) 3.5-10 weight % of a glycol system comprising propylene glycol and a second glycol selected from the group consisting of glycols and polyglycols in which the total amount of propylene glycol in the total gel composition is from 0.2 weight % to 7.5 weight %; and
(b) an external phase comprising:
(i) one or more cyclomethicones having a flash point of 100 degrees C. or less;
(ii) a silicone copolyol;
(iii) a fragrance solubilizer;
(iv) 0-0.45 weight % of an alpha, beta-unsaturated ester malodor counteracting material or mixtures thereof; and
(v) 0-3 weight % of a silicone emollient; wherein: (1) all the amounts are based on the weight of the total formulation, (2) the maximum level of volatile linear silicones is ≦1 weight %, and (3) the external phase is free of silicone emollients that have a refractive index >1.4200.

2. A clear gel as claimed in claim 1 wherein the external phase comprises:
(a) 6.5-23.4 weight % of one or more cyclomethicones having a flash point of 100 degrees C. or less;
(b) 0.6-0.9 weight % on an actives basis of a silicone surfactant having a hydrophilic lipophilic balance ≦8;
(c) 0.1-3.0 weight % of an non-siliconized organic fragrance solubilizer selected from the group consisting of silicone compatible straight or branched hydrocarbons with a molecular weight less than 1000, alkyl substituted phenyl esters with an alkyl carbon chain length between C-1 to C-20, and ethoxylated and or propoxylated ethers with a carbon chain length from C-1 to C-25 and a degree of ethoxylation and/or propoxylation from 1-10;
(d) 0-3 weight % of a low refractive index, <1.4200 at 21 degrees C., silicone emollient excluding (a) and (b) and selected from the group consisting of: (i) volatile linear polydialkylsiloxanes with a flash point is ≦100 degrees C.; (ii) nonvolatile linear polydialkylsiloxanes with a flash point >100 degrees C.; (iii) silanols in which one or two of the alkyl groups of the foregoing materials (i) or (ii) are replaced with a hydroxyl group; and (iv) mixtures of any of the foregoing, provided that the maximum level of volatile linear silicone emollients is ≦1 weight %;
(e) 0-5 weight % fragrance; and
(f) 0.0-0.45 weight % of an alpha, beta-unsaturated ester malodor counteracting material or mixtures thereof.

3. A clear gel as claimed in claim 1 wherein the internal phase comprises:
(a) 14-30 weight % on an anhydrous basis of the glycine-free antiperspirant active salt stabilized by Betaine and comprising either aluminum or aluminum and zirconium metals such that (i) if aluminum and zirconium salt are used then the metal/Cl ratio of the salt is in the range of 0.9-1.3:1; the Betaine/Zr ratio is in the range of 0.2-3.0:1 and the Betaine: aluminum molar ratio is in the range of 0.05-1.0:1; and (ii) if only aluminum salt is used then the aluminum/Cl ratio of the salt is in the range of 0.5-2.5:1; and the Betaine/Al ratio is in the range of 0.05-1.0:1, wherein the Betaine stabilized and/or buffered antiperspirant active salt has a pH in the range of 2-4 when measured in water at a concentration of 15%;
(b) 30-70 weight % water;
(c) 3.5-10 weight % of a water soluble glycol system which comprises (i) at least 0.2% and at most 7.5% propylene glycol and (ii) an additional glycol component selected from the group consisting of ethylene glycol; diethylene glycol; triethylene glycol; tetraethylene glycol; propylene glycol; dipropylene glycol; tripropylene glycol; 1,3 propanediol; 2-methyl propanediol; methyl propanediol; 1,6-hexanediol; 1,3 butanediol; 1,4 butanediol; PEG-4 through PEG-600; PPG-9 through PPG-34; neopentyl glycol; trimethylpropanediol; 2,2 dimethyl-1,3propandiol; 2,2,4,4-tetramethyl-1,3-cyclobutanediol; and mixtures thereof in which the amount of propylene glycol in the glycol system does not exceed 7.5%;
(d) 0-4 weight % of a monovalent or divalent ionizable, water-soluble inorganic or organic salts of the form MₐX_{b},
wherein: a=1 or 2; b=1 or 2;
M is a member selected from the group consisting of Na+1, Li+1, K+1, Mg+2, Ca+2, Sr+2 and Zn; and
X is a member selected from the group consisting of chloride, bromide, iodide, citrate, gluconate, lactate, glycinate, glutamate, ascorbate, aspartate, nitrate, phosphate, hydrogenphosphate, dihydrogenphosphate, formate, maloneate, maleate, succinate, carbonate, bicarbonate, sulfate, and hydrogensulfate;
(e) 0-5% of a water soluble carbon based agent with a refractive index >1.4200 and selected from the group consisting of: glycerin, alanine, lysine, choline, diglycereth-7, triglycereth-7 citrate, glycereth-7 glycolate, glycereth-5 lactate, lauramidopropyl glycerin, glycereth-5 lactate, glycereth-7 glycolate, and glycereth-20 benzoate; and
(f) 0-10 weight % of an alcohol having 2-4 carbons;
wherein:
(i) all amounts are based on the weight of the entire composition,
(ii) the clear gel is an emulsion having a viscosity in the range of 150,000-600,000 centipoise,
(iii) the clear gel has a ratio of oil phase to water phase in the range of 10:90 to 24:76, and
(iv) the composition is free of glycine, borate crosslinkers, soap gelling agents, insoluble suspended materials and secondary water-soluble surfactants having an HLB value ≧9.

4. A clear gel as claimed in claim 2 wherein the internal phase comprises:
(a) 14-30 weight % on an anhydrous basis of the glycine-free antiperspirant active salt stabilized by Betaine and comprising either aluminum or aluminum and zirconium metals such that (i) if aluminum and zirconium salt are used then the metal/Cl ratio of the salt is in the range of 0.9-1.3:1; the Betaine/Zr ratio is in the range of 0.2-3.0:1 and the Betaine: aluminum molar ratio is in the range of 0.05-1.0:1; and (ii) if only aluminum salt is used then the aluminum/Cl ratio of the salt is in the range of 0.5-2.5:1; and the Betaine/Al ratio is in the range of 0.05-1.0:1, wherein the Betaine stabilized and/or buffered antiperspirant active salt has a pH in the range of 2-4 when measured in water at a concentration of 15%;
(b) 30-70 weight % water;
(c) 3.5-10 weight % of a water soluble glycol system which comprises at least 0.2% and at most 7.5% propylene glycol and an additional glycol component selected from the group consisting of ethylene glycol; diethylene glycol; triethylene glycol; tetraethylene glycol; propylene glycol; dipropylene glycol; tripropylene glycol; 1,3 propanediol; 2-methyl propanediol; methyl propanediol; 1,6-hexanediol; 1,3 butanediol; 1,4 butanediol; PEG-4 through PEG-600; PPG-9 through PPG-34; neopentyl glycol; trimethylpropanediol; 2,2 dimethyl-1,3propandiol; 2,2,4,4-tetramethyl-1,3-cyclobutane-diol; and mixtures thereof in which the amount of propylene glycol does not exceed 7.5%;
(d) 0-4 weight % of a monovalent or divalent ionizable, water-soluble inorganic or organic salts of the form MₐX_{b}, wherein
a=1 or 2; b=1 or 2;
M is a member selected from the group consisting of Na⁺ 1, Li+1, K+1, Mg+2, Ca+2, Sr+2 and Zn+2; and
X is a member selected from the group consisting of chloride, bromide, iodide, citrate, gluconate, lactate, glycinate, glutamate, ascorbate, aspartate, nitrate, phosphate, hydrogenphosphate, dihydrogenphosphate, formate, maloneate, maleate, succinate, carbonate, bicarbonate, sulfate, and hydrogensulfate;
(e) 0-5% of a water soluble carbon based agent with a refractive index >1.4200 and selected from the group consisting of: glycerin, alanine, lysine, choline, diglycereth-7, triglycereth-7 citrate, glycereth-7 glycolate, glycereth-5 lactate, lauramidopropyl glycerin, glycereth-5 lactate, glycereth-7 glycolate, and glycereth-20 benzoate; and
(f) 0-10 weight % of an alcohol having 2-4 carbons;
wherein:
(i) all amounts are based on the weight of the entire composition,
(ii) the clear gel is an emulsion having a viscosity in the range of 150,000-600,000 centipoise,
(iii) the clear gel has a ratio of oil phase to water phase in the range of 10:90 to 24:76, and (iv) the composition is free of glycine, borate crosslinkers, soap gelling agents, insoluble suspended materials and secondary water-soluble surfactants having an HLB value ≧9.

5. A clear gel as claimed in claims 1 comprising 9-20 weight % of the one or more cyclomethicones having a flash point of 100 degrees C. or less.

6. A clear gel as claimed in claim 1 comprising 0.5-2 weight % of the non-siliconized organic fragrance solubilizer.

7. A clear gel as claimed in claim 1 comprising 0.1-2.0 weight % of the silicone emollients, other than cyclomethicones, wherein the maximum level of volatile linear silicone emollients is ≦1 weight %.

8. A clear gel as claimed in claim 1 which is free of one or more members selected from the group consisting of volatile linear silicones, non-volatile silicones, waxes, cellulosics, alginates and amino acids.

9. A clear gel as described in claim 1 which further comprises contains 0.1-8.0 weight % of an amino acid or a mixture of amino acids.

10. A clear gel as described in claim 1 comprising at least one amino acid and which is free of one or more members selected from the group consisting of volatile linear silicones, and non-volatile silicones.

11. A clear gel as claimed in claim 1 comprising 17-25 weight % of the antiperspirant active.

12. A clear gel as claimed in any claim 1 comprising 45-65 weight % water.

13. A clear gel as claimed in claim 1 comprising 4-8 weight % of the water soluble glycol system.

14. A clear gel as claimed in claim 1 wherein the additional glycol component is selected from the group consisting of ethylene glycol; diethylene glycol; triethylene glycol; tetraethylene glycol; propylene glycol; dipropylene glycol; tripropylene glycol; 1,3 propanediol; 2-methyl propanediol; methyl propanediol; 1,6-hexanediol; 1,3 butanediol; 1,4 butanediol; PEG-4 through PEG-600; PPG-9 through PPG-34; neopentyl glycol; trimethylpropanediol; 2,2 dimethyl-1,3propandiol; 2,2,4,4-tetramethyl-1,3-cyclobutane-diol; and mixtures thereof.

15. A clear gel as claimed in claim 1 in which the amount of propylene glycol does not exceed 5 weight %.

16. A clear gel as claimed in claim 1 in which the salt is NaCl, CaCl₂ or ZnCl₂.

17. A clear gel as claimed in claim 1 in which the alcohol is 1-8% ethanol.

18. A clear gel as claimed in claim 1 having a viscosity in the range of 200,000-350,000 centipoise.

19. A clear gel as claimed in to claim 1 wherein the refractive index of the gel is between 1.4025 and 1.4150.

20. A clear gel as claimed in claim 1 comprising an aluminum/zirconium salt and wherein the metal/Cl ratio is 0.9-1.1:1; the Betaine/Zr mole ratio is in the range of 0.4-1.5:1; and the Betaine:aluminum molar ratio is in the range of 0.05-0.26:1.

21. A clear gel according to claim 1 wherein the silicone surfactant is a silicone copolyol selected from the group consisting of compounds of
Formulae I and II wherein:
(a) Formula III is:
(R¹⁰)₃—SiO—[(R¹¹)₂—SiO]ₓ—[Si(R¹²)(R^{b}—O—(C₂H₄O)ₚ—(C₃H₆O)ₛ—R^{c})O]_{y}—Si—(R¹³)₃ Formula III
wherein each of R¹⁰, R¹¹, R¹² and R¹³ may be the same or different and each is selected from the group consisting of C1-C6 alkyl; R^{b} is the radical-CₘH₂ₘ—; R^{c} is a terminating radical which is hydrogen, an alkyl group of one to six carbon atoms, an acyl group which is an ester with a terminating alkyl group of 1-4 carbons, or a phenyl group; m has a value of two to eight; p and s have values such that oxyalkylene segment —(C₂H₄O)ₚ—(C₃H₆O)ₛ— has a molecular weight in the range of 200 to 5,000; the oxyalkylene segment has fifty to one hundred mole percent of oxyethylene units —(C₂H₄O)ₚ— and one to fifty mole percent of oxypropylene units —(C₃H₆O)ₛ—; x has a value of 8 to 400; and y has a value of 2 to 40;
(b) Formula IV is:
(R¹⁰)₃—SiO—[(R¹¹)₂—SiO]ₓ—[Si(R¹²)(R^{b}—O—(C₂H₄O)ₚ—R^{c})O]_{y}—Si—(R¹³)₃ Formula IV
wherein p has a value of 6 to 16; x has a value of 6 to 100; and y has a value of 1 to 20 and other moieties have the same definition as defined in Formula III; and
(c) alternate embodiments of both Formulae III and IV wherein the linking group R^{b}, the oxyalkylene segments, and the terminating radical R^{c} occupy positions bonded to the ends of the siloxane chain and are not bonded to a silicon atom in the siloxane chain.

22. A clear gel as claimed in claim 1 wherein the non-siliconized fragrance solubilizer is selected from the group consisting of PPG-3 myristyl ether, hydrogenated polyisobutene, and C12-15 alkyl benzoate.

23. A clear gel as claimed in claim 1 additionally comprising 0.1-1.0 weight % of a silicone emollient other than cyclomethicones.

24. A clear gel as claimed in claim 1 comprising:
(a) 17-23 weight % of an external phase comprising
(i) 8-20 weight % of one or more of the cyclomethicones;
(ii) 0.6-0.8 weight % of the silicone surfactant;
(iii) 0.5-2 weight % of the fragrance solubilizer;
(iv) 0-1 weight % of the silicone emollient;
(v) 0.0-0.45 weight % weight percent of an odor neutralizing alpha, beta unsaturated ester mixture; and
(vi) 0-1.0 weight % of a fragrance; and
(b) 77-83 weight % of an internal phase comprising:
(i) 17-25 weight % on an anhydrous basis of the antiperspirant active salt;
(ii) 4-8% weight % of the water soluble glycol system which comprises at least 0.2% propylene glycol and an additional glycol component selected from the group consisting of dipropylene glycol, tripropylene glycol, 2-methyl 1,3 propanediol, 1,3 propanediol, methyl propylene glycol, PEG 400, and low molecular weight polyethylene glycol;
(iii) 2-8 weight % ethanol or propanol; and
(iv) 0.8-3 weight % of the monovalent or divalent ionizable, water soluble inorganic or organic salt;
wherein the amounts are based on the weight of the entire composition.

25. A clear gel as claimed in claim 1 wherein the fragrance level is less than 0.1 weight % and the alpha, beta-unsaturated ester malodor counteracting material is between 0.1 and 0.4 weight %.

26. A clear gel as claimed in claim 1 wherein the fragrance level is between 0.1 and 2.0% and the alpha, beta-unsaturated ester malodor counteracting material is between 0.1 and 0.4%

27. A clear gel as claimed in claim 1 comprising an alpha, beta-unsaturated ester malodor counteracting material comprising a combination of two or more members selected from the following groups:
(a) 3-phenyl-2-propenoic acid alkyl esters wherein R¹ is a substitutent on the benzene ring and is an alkyl, an alkoxy, an aryl, or a substituted aryl; and R² is a subsistent group replacing the carboxylic acid hydrogen to form the ester where R² has greater than 6 carbon atoms, an aryl, or a substituted aryl group, preferably, W is a C6 to C12 alkyl or is a benzyl group; and
(b) an ester of fumaric or maleic acid having linear ester carbon chains from 3-9 carbons.

28. A clear gel as claimed in claim 1 comprising: (a) an alpha, beta-unsaturated ester malodor counteracting material comprising an e-phenyl propenoic acid ester selected from the group consisting of octyl methoxy cinnamate, phenylethyl cinnamate, and benzyl cinnamate; and (b) an aliphatic unsaturated ester selected to be dihexyl fumarate.

29. A clear gel as claimed in claim 1 comprising a combination of two or more members selected from the group consisting of:
(a) 3-phenyl-2-propenoic acid alkyl esters wherein R¹ is a substitutent on the benzene ring and is an alkyl, an alkoxy, an aryl, or a substituted aryl; and R² is a substitutent group replacing the carboxylic acid hydrogen to form the ester where R² is a C6 to C12 alkyl or is a benzyl group; and
(b) an ester of fumaric or maleic acid having linear ester carbon chains from 3-9 carbons.

30. A clear gel as claimed in claim 1 wherein the alpha, beta unsaturated ester malodor counteracting material is a mixture of (a) an e-phenyl propenoic acid ester selected from the group consisting of octyl methoxy cinnamate, phenylethyl cinnamate and benzyl cinnamate and (b) dihexyl fumarate.
